(19) United States
(12) Patent Application Publication
(10) Pub. No.: US 2021/0386278 A1
(43) Pub. Date: Dec. 16, 2021

(54) ENDOSCOPE FLUORESCENCE INSPECTION DEVICE

(71) Applicant: Mediators Inc., Minneapolis, MN (US)

(72) Inventors: Mark Jackson, Great Wakering (GB); Joshua J. Korth, St. Louis Park, MN (US); Huyen Bui, Brooklyn Park, MN (US); Tuan Nguyen, Chaska, MN (US); Mer Win Cheong, Pomezia (IT); Nicholas G. Bromiley, Annandale, MN (US)

(21) Appl. No.: 17/289,493

(22) PCT Filed: Nov. 1, 2019
(86) PCT No.: PCT/US2019/059364

§ 371 Date: Apr. 28, 2021

Related U.S. Application Data
(60) Provisional application No. 62/755,854, filed on Nov. 5, 2018.

(51) Int. Cl. A61B 1/04; A61B 1/00; A61B 1/07; A61B 1/06; A61B 1/12

(52) U.S. Cl. CPC A61B 1/043; A61B 1/00059; G01N 21/94; A61B 1/0684; A61B 1/121; A61B 1/07

(57) ABSTRACT

Embodiments of a system and method of use for endoscope fluorescence inspection are generally described herein. In an example embodiment, a fluorescence inspection device provides capabilities for internal inspection of an endoscope lumen through a handheld unit coupled to a light pipe that identifies fluorescence from residual biological material exposed to a fluorescing agent. In another example embodiment, a fluorescence inspection device provides capabilities for external inspection of an endoscope surface through a handheld unit which emits excitation light and identifies fluorescence of residual biological material exposed to the fluorescing agent. The embodiments may also include an output device to output an indication in response to a detection (or lack of detection) of the fluorescent light with the fluorescence inspection device. Additional use examples and device structures are also described.

### PRIORITY CLAIM

This application claims priority to and the benefit of U.S. Provisional application with Ser. No. 62/755,854, filed on Nov. 5, 2018, entitled ENDOSCOPE FLUORESCENCE INSPECTION DEVICE, which is herein incorporated by reference in its entirety.

### TECHNICAL FIELD

Embodiments pertain to the cleaning and reprocessing of reusable medical equipment. Some embodiments more specifically relate to an inspection device and inspection techniques using fluorescence for the detection of residual biological materials.

### BACKGROUND

Specific de-contamination procedures and protocols are utilized to clean reusable medical equipment. As one example in the medical setting involving reusable medical equipment, endoscopes that are designed for use in multiple procedures must be fully cleaned and reprocessed after a medical imaging procedure to prevent the spread of infectious organisms. Once an endoscope is used in the medical procedure, an endoscope is considered contaminated until it is properly cleaned and disinfected through a series of specific cleaning actions.

A number of protocols and assisting equipment for cleaning, disinfection, and inspection are used by current medical practices to reprocess endoscopes and prepare them for subsequent procedures. For example, various machines and devices such as automated endoscope reprocessors are used to perform deep cleaning of an endoscope, through the application of disinfecting cleaning solutions. High-level disinfection or sterilization processes are typically performed after manual cleaning to remove any remaining amounts of soils and biological materials. However, an endoscope is not considered as ready for high-level disinfection or sterilization until it has been inspected and verified to function correctly, without any damage or leaking parts. If the endoscope includes damaged surfaces, leaks, broken controls, or the like, the endoscope may not be fully exposed to deep cleaning by the disinfecting chemicals, and the opportunity for spreading contamination significantly increases.

During existing manual cleaning procedures, a human technician may inspect the endoscope for damage and perform various types of inspections, verifications, or tests on external surfaces and operational the components of the endoscope. However, many types of contaminants and damage within or on the endoscope are not readily visible or observable by a human. Therefore, there is a need to improve cleaning processes of endoscopes to reduce the incidence and amount of residual biological material, as well as a need to improve inspection processes to detect residual biological material or damage to the endoscope.

### BRIEF DESCRIPTION OF THE DRAWINGS

FIG. 1 illustrates an overview of devices and systems involved in stages of endoscope use and reprocessing, according to various examples discussed herein;

FIG. 2 is a schematic cross-section illustration of an endoscope, operated according to various examples discussed herein;

FIG. 3 illustrates data flows provided with a cleaning workflow and tracking system, during respective stages of endoscope use and processing, according to various examples discussed herein;

FIG. 4 is a block diagram of system components used to interface among tracking and inspection systems and devices according to various examples discussed herein;

FIG. 5 is a use illustration of a fluorescence inspection device, as configured according to various examples discussed herein;

FIG. 6 is a block diagram that illustrates components and integration of a fluorescence inspection system, according to various examples discussed herein; and

FIG. 7 illustrates a flowchart for a method of endoscope inspection, according to various examples discussed herein.

### DETAILED DESCRIPTION

The following description and the drawings sufficiently illustrate specific embodiments to enable those skilled in the art to practice them. Other embodiments may incorporate structural, logical, electrical, process, and other changes. Portions and features of some embodiments may be included in, or substituted for, those of other embodiments. Embodiments set forth in the claims encompass all available equivalents of those claims.

Conventional techniques for endoscope cleaning involve various types and levels of inspection. For instance, national guidelines for endoscope cleaning recommend visual inspection of the endoscope, in addition to the use of protein and carbohydrate detection, to assess the level of cleanliness achieved. While a number of techniques have been developed and promoted for the detection of residual contamination, the levels of detection and the ease of use of these techniques are variable and inconsistent in many real-world settings.

Various device configurations and techniques are described herein for the improvement of endoscope cleaning and handling processes using fluorescence detection. Specifically, the detection of fluorescence emitted from biological materials may be used to identify residual contamination on an endoscope or in particular endoscope components. Such fluorescence may be triggered, identified, and detected as part of manual or automated actions occurring in an endoscope cleaning workflow.

In an example, a fluorescence scanner is provided in a form factor that allows simple detection of fluorescing effects emitted from biological materials on, or within, an endoscope. This fluorescence may be detected from effects emitted in the presence of a fluorescing agent, such as proteins, which react with a fluorescing cleaning or rinsing agent to produce fluorescence in the presence of a particular light wavelength. Beneficially, this fluorescence may be triggered from biofilms and complex biological material deposits that may resist cleaning or evade detection by conventional manual cleaning processes.

Endoscope suction channels are particularly susceptible to the buildup of biofilm if inadequately cleaned due to the high levels of body fluids they carry during an endoscopic procedure including, but not limited to, blood, tissue, feces, bile, etc. The external surfaces of the endoscope may also propagate biofilm unbeknown and undetected by the user. Such biofilm may not be easily detected or removed in either automated cleaning machines or with manual human inspection. Thus, the use of fluorescence detection with the presently described device configurations and techniques enables the identification of such conditions, and the verification of remediation for such conditions.

Also in an example, the fluorescence scanner is adapted to allow coupling with an optical fiber that allows examination of hard-to-access spaces of the endoscope. Specifically, the optical fiber may be of a particular size and shape (e.g., length and diameter) and use characteristics (e.g., flexible, including one or more individual fibers in a fiber bundle) to traverse though the relatively narrow internal endoscope channels and examine the state of a biopsy channel (a portion of the suction channel), or other air, water, or access channels for fluorescence.

Also in an example, the fluorescence scanner is adapted as a handheld device form factor that can be used to independently scan the external surfaces of the endoscope for contamination. The fluorescence scanner may be used to identify areas or sections of an endoscope which remain contaminated, with an easy-to-observe indication that the inspected area includes detectable levels of fluorescence from biological agents.

Further aspects of the scanner, inspection system, and associated techniques discussed herein enable integration of fluorescence detection into endoscope inspection and cleaning verification processes. This may include an output screen, data input facility, and recording/download facility, integrated into or operably coupled to the fluorescence inspection system. Further aspects also include the integration of endoscope identification with the disclosed device, system, and processing techniques, such as capturing a radio frequency identifier (RFID) or bar code for identification of the endoscope, tracking a contamination status from results of fluorescence detection on the endoscope, and tracking the performance of the fluorescence detection along with other verification or quality assurance activities. Other aspects of use within an endoscope cleaning, handling, and tracking workflow(s) will be apparent from the following examples.

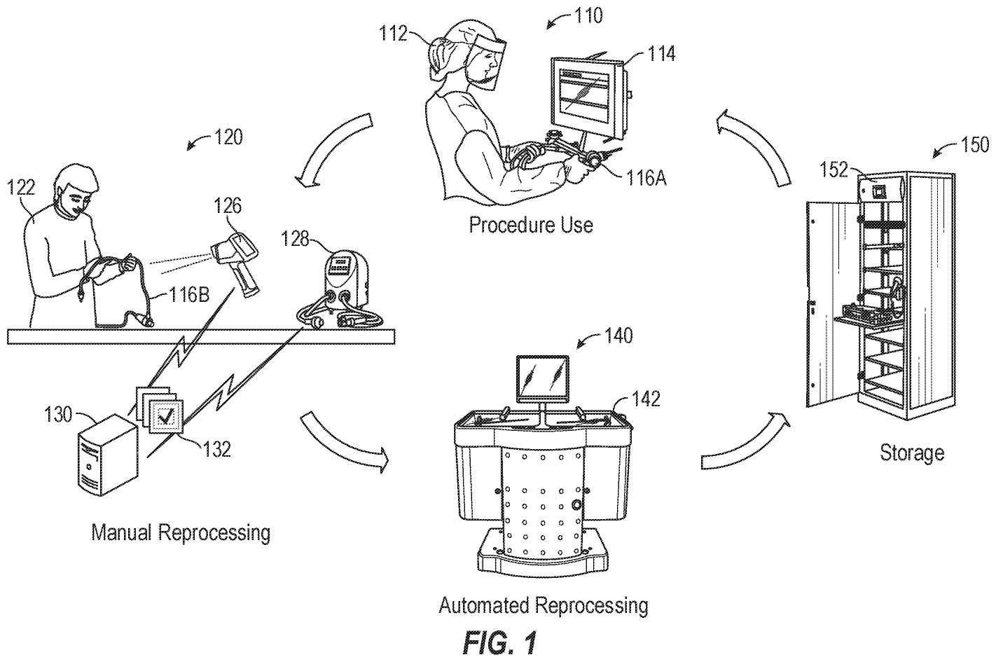

FIG. 1 illustrates an overview of devices and systems involved in example stages of endoscope use and reprocessing. In the environment illustrated in FIG. 1, a series of stages are sequentially depicted for use and handling of the endoscope, transitioning from a procedure use stage 110, to manual reprocessing stage 120, to an automated reprocessing stage 140, to a storage stage 150. It will be understood that the stages 110, 120, 140, 150 as depicted and described provide a simplified illustration of typical scenarios in the use, handling, and reprocessing for reusable endoscopes. As a result, many additional steps and the use of additional devices and procedures (or, substitute procedures and substitute devices) may be involved in the respective stages.

The procedure use stage 110 depicts a human user 112 (e.g., technician, nurse, physician, etc.) who handles an endoscope. At the commencing of the procedure use stage 110, the endoscope 116A is obtained in a sterile or high-level disinfected/clean state. This disinfected/clean state typically results from reprocessing and storage of the endoscope 116A, although the state may also be provided from a sterilized repair or factory-provided state. In the procedure use stage 110, the endoscope 116A may be used for various endoscopic procedures (e.g., colonoscopy, upper endoscopy, etc.) on a subject human patient, for any number of diagnostic or therapeutic purposes. During the endoscopic procedures, the endoscope 116A is exposed to biological material from the subject patient or the surrounding environment. Thus, at the completion of the procedure use stage 110, the endoscope 116A exists in a contaminated state.

The disinfected or contamination state of the endoscope 116A may be tracked by a tracking system for purposes of monitoring, auditing, and other aspects of workflow control. An interface 114 to the tracking system is shown, which receives an identifier of the endoscope 116A and provides a graphical status as output. The tracking system may be used in the procedure use stage 110 (and the other stages 120, 140, 150) to identify the use of the endoscope 116A to be associated with a particular imaging procedure, patient, procedure equipment, procedure room, preparation or cleaning protocol, or other equipment or activities. This identifying information may enable the tracking system to track the contamination or disinfected state of the endoscope, and to identify and prevent exposure of contamination or infectious agents to patients or handling personnel from damaged endoscopes or improper cleaning procedures.

After the procedure use stage 110, the endoscope transitions to handling in a manual reprocessing stage 120. The manual reprocessing stage 120 specifically depicts the use of manual cleaning activities being performed by a technician 122, to clean the endoscope 116B. The type of manual cleaning activities may include disassembly and removal of components, applying brushes to clear channels, wiping to remove visible liquids and solids, and other human-performed cleaning actions. Some of the manual cleaning activities may occur according to a regulated sequence or manufacturer-specified instructions.

The manual reprocessing stage 120 also depicts the use of a flushing aid device 128 and a fluorescence inspection device 126 to conduct additional aspects of cleaning and inspection. In an example, the flushing aid device 128 serves to perform an initial chemical flush of the internal channels of the endoscope 116B (e.g., water, air, or suction channels) with cleaning agents. The flushing aid device 128 may also enable the performance of leak testing, to verify whether components or structures of the endoscope leak fluid (e.g., leak water or air). In other examples, the flushing or leak test actions performed by the flushing aid device 128 are manually performed by the syringing of chemicals or air into the endoscope channels. The results of the leak testing and the flushing may be tracked or managed as part of a device tracking or cleaning workflow, such as by communicating such results 132 to a tracking computing system 130.

The use of fluorescence inspection with the fluorescence detection device 126 may involve aspects of inspection and verification of a non-contaminated (clean) state of the endoscope 116B. Such fluorescence inspection may include: a scan of an external surface of the endoscope 116b (e.g., an insertion tube, control, etc.) performed with a fluorescence scanner integrated into the fluorescence detection device 126; an inspection of an internal component of the endoscope (e.g., a lumen of a channel) performed with a light pipe or other accessory (not shown) connected to the fluorescence detection device 126; and other emission of light to trigger fluorescence and detect such fluorescence of biological material exposed to a fluorescing agent. The results of the fluorescence inspection may include a detection or non-detection of a particular state (e.g., clean, contaminated) for respective components of the endoscope 116B (e.g., a particular surface, channel, etc.). Such results may be tracked or managed as part of the device tracking or cleaning workflow, including communicating such results 132 to the tracking computing system 130. Further details on the fluorescence inspection process and use cases in which fluorescence may be deployed are discussed in more detail in the examples below.

In an example, a borescope inspection system (not shown) may also be integrated into aspects of the inspection process, such as to visually inspect an interior lumen of a channel in the endoscope for damage, contamination, blockage, etc. A borescope inspection process may occur before or after the performance of the leak test, flushing, fluorescence inspection, or other cleaning or testing activities in the manual reprocessing stage 120. The imaging data obtained with the borescope may be used for human or machine-based analysis and examination of the condition of a lumen and its associated channel. For example, the imaging data may be analyzed in connection with a trained artificial intelligence (e.g., machine learning) model to analyze image data from the borescope and identify a state of the endoscope channel.

In specific examples, the detection of fluorescence may be coordinated with or integrated with this image capture or other uses of a borescope. For example, a light pipe connected to a fluorescence emitter and detector (e.g., provided from fluorescence detection device 126), may be terminated at a tip of a borescope to emit and receive light in a lumen for fluorescence detection purposes. As another example, the borescope may include components of the presently described fluorescence device, such as an emitter or sensor hosted directly within the borescope, for purposes of detecting residual biological materials via fluorescence while also inspecting a lumen for damage via visible light.

After completion of the manual reprocessing stage 120, the endoscope is handled in an automated reprocessing stage 140. This may include the use of an automatic endoscope reprocessor (AER) 142, or other machines which provide a high-level disinfection and sterilization of the endoscope. For instance, the AER 142 may perform disinfection for a period of time (e.g., for a period of minutes) to expose the interior channels and exterior surfaces of the endoscope to deep chemical cleaning and disinfectant solutions. The AER 142 may also perform rinsing procedures with clean water to remove chemical residues.

After completion of the automated reprocessing stage 140 and the production of the endoscope in a disinfected state, the endoscope transitions to handling in a storage stage 150. This may include the storage of the endoscope in a sterile storage unit 152. In some examples, this stage may also include the temporary storage of the endoscope in a drying unit. Finally, retrieval of the endoscope from the storage stage 150 for use in a procedure results in transitioning back to the procedure use stage 110.

The overall cleaning workflow provided for an endoscope within the various reprocessing stages 120 and 140 may vary according to the specific type of device, device-specific requirements and components, regulations, and the types of cleaning chemicals and devices applied. However, the overall device use and cleaning workflow, relative to stages of contamination, may be generally summarized in stages 110, 120, 140, 150, as involving the following steps:

1) Performance of the endoscopic procedure. As will be well understood, the endoscopic procedure results in the highest amount of contamination, as measured by the amount of microbes contaminating the endoscope.

2) Bedside or other initial post-procedure cleaning. This cleaning procedure removes or reduces the soils and biological material encountered on the endoscope during the endoscopic procedure. As a result, the amount of contamination, as measured by the amount of microbes, is reduced.

3) Transport to reprocessing. The more time that is spent between the procedure and reprocessing results in a potential increase in the amount of contamination or difficulty to remove the contamination, due to biological materials drying, congealing, growing, etc.

4) Performance of a leak test (e.g., conducted in the manual reprocessing stage 140 with the flushing aid device 128 or a standalone leak testing device or procedure (not shown)). This leak test is used to verify if any leaks exist within channels, seals, controls, valve housings, or other components of the endoscope. If the endoscope fails the leak test, or encounters a blockage during flushing, then high-level disinfection or sterilization attempted in automated reprocessing will be unable to fully flush and disinfect all areas of the endoscope. Further, if the leak test fails but the instrument is placed in an automatic reprocessing machine, the instrument will be damaged through fluid ingress during the reprocessing cycle.

5) Manual washing (e.g., conducted in the manual reprocessing stage 140 with brushes, flushing, etc.). This aspect of manual washing is particularly important to remove biofilm and lodged biological agents from spaces on or within the endoscope. Biofilm generally refers to a group of microorganisms that adheres to a surface, which may become resistant or impervious to cleaning and disinfectant solutions. The successful application of manual washing significantly reduces the amount of contamination on the endoscope.

6) Residual contamination inspection (e.g., conducted in manual reprocessing stage 140 with a fluorescence inspection system). Microbes and in particular biofilm may resist cleaning if lodged in damaged or irregular portions of the endoscope. A procedure of manual or human-guided inspection for residue can be used to identify an abnormal state (e.g., a compromised, contaminated state) caused by the presence of biological materials (such as biofilms) within the interior channels, exterior surfaces, or components of the endoscope. Such damage inspection may be performed or confirmed by use of a fluorescence inspection system, fluorescence devices and fluorescence inspection techniques, borescope inspection system, visual inspection system, and other mechanisms discussed herein.

7) High level disinfection or sterilization (e.g., conducted in AER 142). Upon successful conclusion of the high-level disinfection or sterilization process, in an ideal state for an endoscope with no damage, no biological contamination will remain from the original endoscopic procedure. In an example, the presently described techniques for detection of fluorescence may be integrated with high level disinfection or sterilization processes, including the detection of fluorescence from biological materials remaining on surfaces, in rinsing fluids, and the like.

8) Rinse and Air Purge. This stage involves the introduction of clean water and air, to flush any remaining chemical solution and to place the endoscope in a disinfected and clean state. The risk of introducing new contamination may be present if contaminated water or air are introduced to the endoscope.

9) Transport to Storage. This stage involves the transport from the AER or other device to storage. A risk of introducing new contamination may be present based on the method and environment of transport and handling.

10) Storage. This stage involves the storage of the endoscope until needed for a procedure. A risk of introducing new contamination may be present based on the conditions in the storage unit.

11) Transport to Patient. Finally, the endoscope is transported for use in a procedure. A risk of introducing new contamination may also be present based on the method and environment of transport and handling.

Further aspects which may affect contamination may involve the management of valves and tubing used with a patient. For instance, the use of reusable valves, tubing, or water bottles in the procedure may re-introduce contamination to the endoscope. Accordingly, the disinfected state of a processed endoscope can only be provided in connection with the use of other sterile equipment and proper handling in a clean environment. The use of fluorescence detection may also be adapted to the verification of a lack of contamination from any of such states, in connection with cleaning, rinsing, and other forms of fluorescence agents.

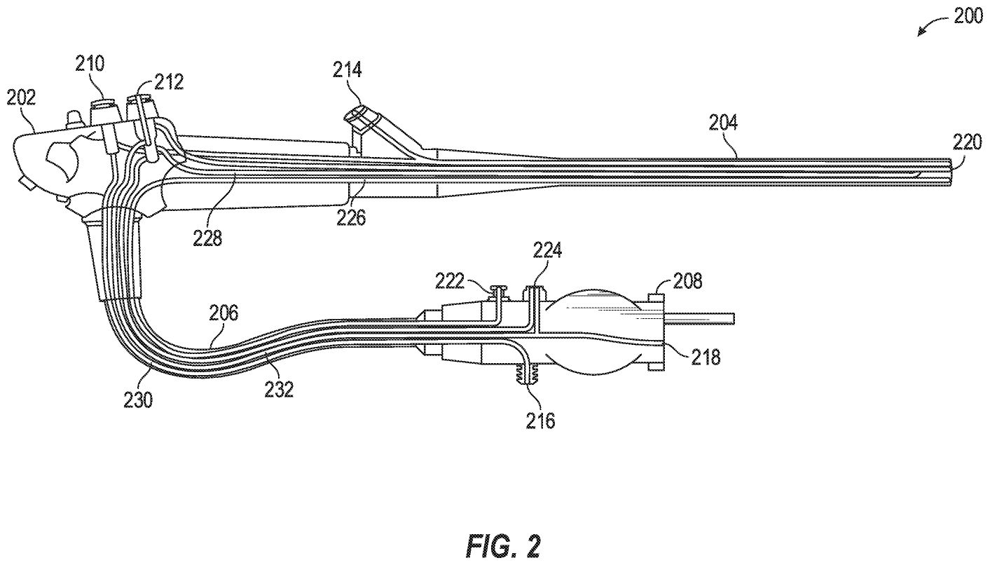

FIG. 2 is a schematic cross-section illustration of an endoscope 200, operable according to various examples. The endoscope 200 as depicted includes portions that are generally divided into a control section 202, an insertion tube 204, a universal cord 206, and a light guide section 208. A number of imaging, light, and stiffness components and related wires and controls used in endoscopes are not depicted for simplicity. Rather, FIG. 2 is intended to provide a simplified illustration of the channels important for endoscope cleaning workflows. It will be understood that the presently discussed endoscope cleaning workflows will be applicable to other form factors and designs of endoscopes. The techniques, systems, and apparatus discussed herein can also be utilized for inspection operations on other instruments that include lumens that can become contaminated or damaged during use.

The control section 202 hosts a number of controls used to actuate the positioning, shape, and behavior of the endoscope 200. For instance, if the insertion tube 204 is flexible, the control section 202 may enable the operator to flex the insertion tube 204 based on patient anatomy and the endoscopic procedure. The control section 202 also includes a suction valve 210 allowing the operator to controllably apply suction at a nozzle 220 via a suction channel 230. The control section 202 also includes an air/water valve 212 which allows the distribution of air and/or water from an air channel 232 (provided from an air pipe source 218) or a water channel 228 (provided from a water source connected to a water source connector 224) to the nozzle 220. The depicted design of the endoscope 200 also includes a water jet connector 222 via a water-jet channel 226, to provide additional distribution of water separate from the air channel 232.

The universal cord 206 (also known as an “umbilical cable”) connects the light guide section 208 to the control section 202 of the endoscope. The light guide section 208 provides a source of light which is distributed to the end of the insertion tube 204 using a fiber optic cable or other light guides. The imaging element (e.g. camera) used for capturing imaging data may be located at in the light guide section 208 or adjacent to the nozzle 220 (at the distal end of the insertion tube).

As shown, the various channels of the endoscope 200 allow the passage of fluids and objects, which may result in the contamination throughout the extent of the channels. The portion of the suction channel 230 which extends from the biopsy valve 214 to the distal end of the insertion tube 204 (to the nozzle 220) is also known as the biopsy channel. In particular, the biopsy channel, and the remainder of the suction channel 230, is subject to a high likelihood of contamination and/or damage in the course of an endoscopic procedure. For example, the insertion, manipulation, and extraction of instruments (and biological material attached to such instruments) through the suction channel 230 commonly leads to the placement of microbes within the suction channel 230.

Any damage to the interior layer(s) of the biopsy channel, such as in scratches, nicks, or other depressions or cavities to the interior surface caused by instruments moving therein may also lead to deposits of biological material. Such biological material which remains in cavities, or which congeals in the form of biofilm, may be resistant to many manual cleaning techniques such as brushes pulled through the suction channel. Such damage may also occur in the other channels 228, 230, 232, as a result of usage, deterioration, or failure of components. The techniques discussed herein provide enhanced techniques in connection with the inspection and verification of the integrity of the channels 228, 230, 232, and specifically the integrity from deposited biological materials and contamination in such channels 228, 230, 232.

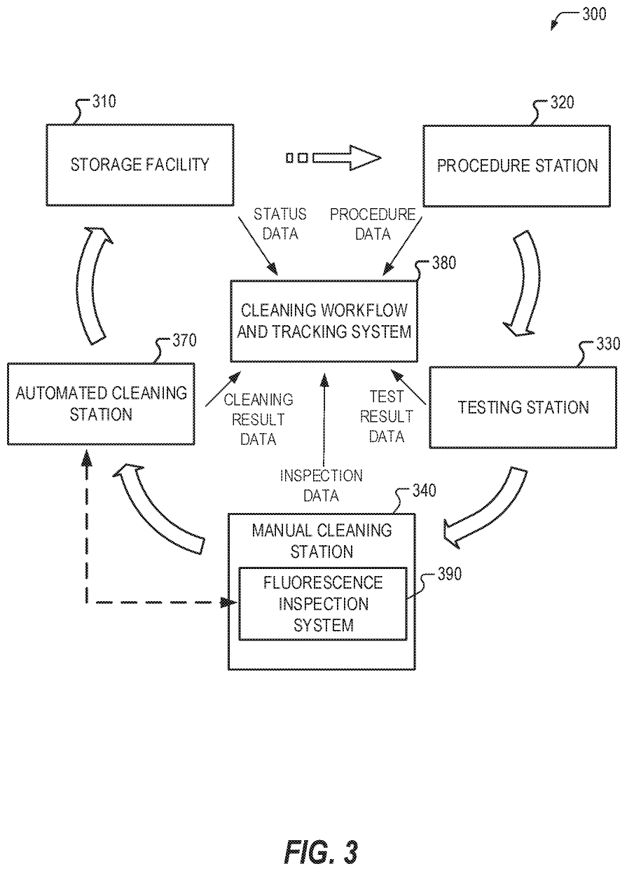

FIG. 3 illustrates data flows 300 provided with an example cleaning workflow and tracking system 380, during respective stages of endoscope use and processing, including the use of a fluorescence inspection system 390 used to perform an integrity verification of one or more endoscope channels or surfaces. Other types of inspection and cleaning systems, such as a borescope inspection system and visual inspection processing system, are not illustrated but may also be integrated as part of the data flows 300.

The data flows 300 illustrate the generation and communication of data as an endoscope is handled or used at various locations. These include: status of the endoscope at a storage facility 310 (e.g., the storage unit 152 in the storage stage 150), as indicated via status data (e.g., a location and sterilization status of the endoscope); status of the use of the endoscope at a procedure station 320 (e.g., as handled in the procedure use stage 110), as indicated via procedure data (e.g., an identification of a patient, physician, and handling details during the procedure); status of the testing of the endoscope at a testing station 330 (e.g., at a leak or component test device), as indicated via test result data (e.g., a pass or fail status of a test, measurement values, etc.); status of the manual cleaning actions performed at a manual cleaning station 340 (e.g., as performed by the technician 122), as indicated by inspection data (e.g., a status that logs the timing and result of inspection procedures, cleaning activities, etc.); and a status of the machine cleaning actions performed at an automated cleaning station 370 (e.g., as performed by the AER 124), as indicated by cleaning result data (e.g., a status that logs the procedures, chemicals, timing of automated reprocessing activities). Such statuses and data may be communicated for storage, tracking, maintenance, and processing, at a cleaning workflow and tracking system 380 (and databases operated with the system 380).

The location of the endoscope among the stations, and activities performed with the endoscope, may be performed in connection with a specific device handling workflow. Such a workflow may include a step-by-step cleaning procedure, maintenance procedures, or a tracking workflow, to track and manage a disinfected or contaminated status, operational or integrity status, or cleaning procedure status of the endoscope components or related equipment. In connection with cleaning operations at the manual cleaning station 340 or the automated cleaning station 370, the subject endoscope may be identified using a tracking identifier unique to the endoscope, such as a barcode, RFID tag, or other identifier coupled to or communicated from the endoscope. For instance, fluorescence inspection system 390 may host an identifier detector to receive identification of the particular endoscope being cleaned at the respective cleaning station. In an example, the identifier detector comprises a RFID interrogator or bar code reader used to perform hands-free identification.

Additionally, in connection with a cleaning workflow, tracking workflow, or other suitable device handling workflow, a user interface may be output to a human user via a user interface device (e.g., a display screen, audio device, or combination). For example, the user interface may request input from the human user to verify whether a particular cleaning protocol has been followed by the human user at each of the testing station 330, manual cleaning station 340 and automated cleaning station 370. A user interface may also output or receive modification of the status in connection with actions at the storage facility 310 and the procedure station 320. The input to such user interface may include any number of touch or touch-free (e.g., gesture, audio command, visual recognition) inputs, such as with the use of touchless inputs to prevent contamination with an input device.

In various examples, input recognition used for control or identification purposes may be provided within logic or devices of any of the stations 310, 320, 330, 340, 370, or as interfaces or controls to the fluorescence inspection system 360. In still further examples, tracking of patients, cleaning personnel, technicians, and users or handlers of the endoscope may be tracked within the data values communicated to the cleaning workflow and tracking system 380. The interaction with the cleaning workflow and tracking system 380 may also include authentication and logging of user identification information, including validation of authorized users to handle the device, or aspects of user-secure processing.

A variety of inquiries, prompts, or collections of data may occur at various points in a device cleaning or handling workflow, managed by the cleaning workflow and tracking system 380, to collect and output relevant data. Such data may be managed for procedure validation or quality assurance purposes, for example, to obtain human verification that a cleaning process has followed proper protocols, or that human oversight of the cleaning process has resulted in a satisfactory result. Workflow steps may also be required by the workflow and tracking system 380 to be performed in a determined order to ensure proper cleaning, and user inquiries and prompts may be presented in a determined order to collect full information regarding compliance or procedure activities. Further, the cleaning workflow and tracking system 380 may be used to generate an alert or display appropriate prompts or information if a user or device does not fully completion certain steps or procedures.

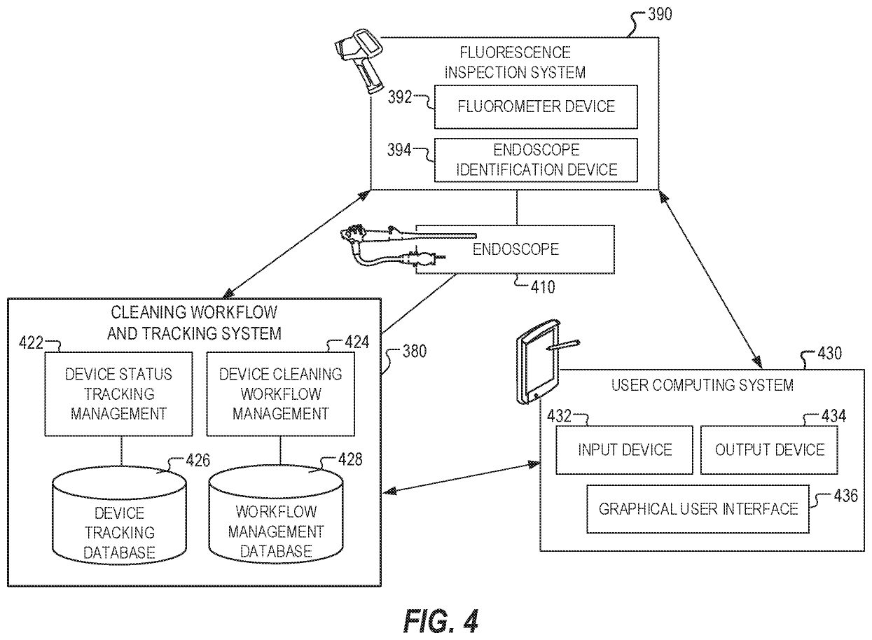

FIG. 4 is a block diagram of system components used to interface among example imaging, tracking, and processing systems. As shown, the components of the fluorescence inspection system 390 may include a fluorometer device 392 and an endoscope identification device 394. The fluorometer device 392 may determine and provide a status of detection of fluorescence (e.g., a detection of fluorescing biological materials) as an output from the system 390 or as a value provided to the cleaning workflow and tracking system 380. This status of detection may be determined from and tracked for the inspection of subject areas (e.g., internal channels, external surfaces) of the endoscope 410 or a component of the endoscope 410. The use of the fluorescence inspection system 390 may be tracked and managed as part of an inspection procedure in a cleaning workflow, with resulting tracking and inspection data maintained by the cleaning workflow and tracking system 380.

The cleaning workflow and tracking system 380 may include functionality and processing components used in connection with a variety of cleaning and tracking purposes involving the endoscope 410. Such components may include device status tracking management functionality 422 that utilizes a device tracking database 426 to manage data related to status(es) of contamination, damage, tests, and usage for the endoscope 410 (e.g., among any of the stages 110, 120, 140, 150). Such components may also include a device cleaning workflow management functionality 424 used to track cleaning, testing, verification activities, initiated as part of a cleaning workflow for the endoscope 410 (e.g., among the reprocessing stages 120, 140). As specific examples, the workflow management database 428 may log the timing and performance of specific manual or automatic cleaning actions, the particular amount or type of cleaning or disinfectant solution applied, which user performed the cleaning action, and the like.

The data and workflow actions in the cleaning workflow and tracking system 380 may be accessed (e.g., viewed, updated, input, or output) through use of a user computing system 430, such as with an input device 432 and output device 434 of a personal computer, tablet, workstation, or smartphone, operated by an authorized user. The user computing system 430 may include a graphical user interface 436 to allow access to the data and workflow actions before, during, or after any of the handling or cleaning stages for the endoscope 410 (e.g., among any of the stages 110, 120, 140, 150). For instance, the user computing system 430 may display a real-time status of whether the endoscope 410 is disinfected, which tests have been completed and passed during cleaning, and the like. Additionally, the user computing system 430 may communicate data directly or indirectly with the fluorescence inspection system 390, including in scenarios where the fluorescence inspection system 390 is used independently of the cleaning workflow and tracking system.

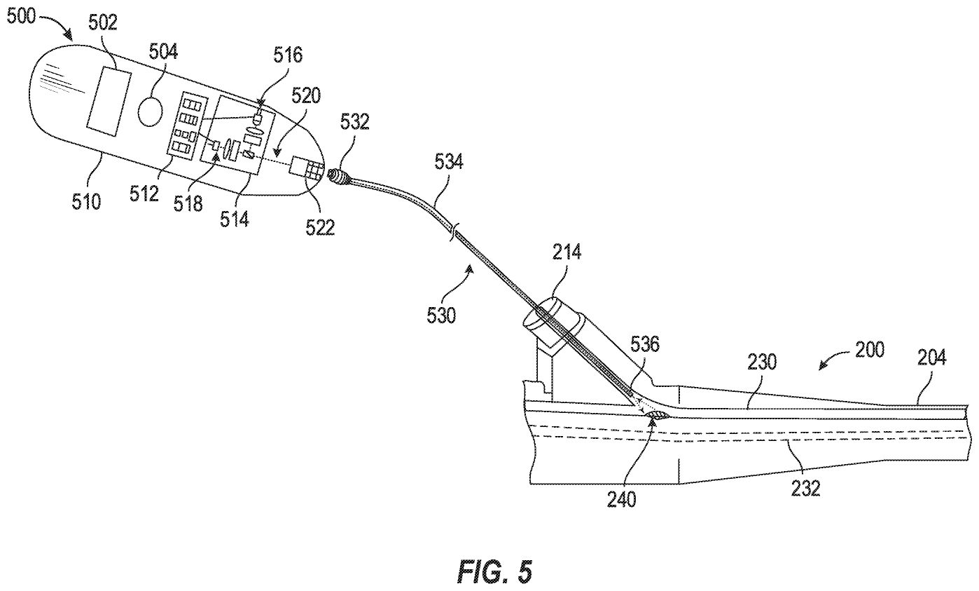

FIG. 5 is an example use illustration of a fluorescence inspection device 500, configured to perform the examination of a lumen as illustrated within a cross-section of the endoscope 200. Only a portion of the endoscope 200 and a limited number of the components of the endoscope 200 are depicted for simplicity. However, it will be understood that the examination of other lumens, channels, components, and areas of an endoscope or like equipment may be facilitated by the fluorescence inspection device 500 and associated techniques.

The use illustration of FIG. 5 specifically depicts the fluorescence inspection device 500 in a handheld form factor provided from a housing 510, while illustrating a cutaway to internal components including: an indication device 502 (e.g., LED array or LCD screen) to output a measurement (e.g., numerical value) or detection state (e.g., a detected state indicator or a non-detected state indicator) of fluorescence; a control 504 (e.g., button) used to actuate operation and functions of the inspection device 500; a circuit board 512 including circuitry for control of the operation and functions of the inspection device 500; a fluorometer 514 used for emitting excitation light and sensing fluorescence light; and a connector 522 exposed from the housing 510 that is usable to removably mate and couple with a connector 532 of a light pipe 530. In an example, the fluorometer 514 includes a light emitter 516 (e.g., a light emitting diode) configured to emit light at a fluorescence excitation wavelength (possibly among other wavelengths or as part of a continuous spectrum that includes the fluorescence excitation wavelength). In an example, the fluorometer 514 also includes a light sensor 518 (e.g., a detector or photosensor) configured to sense light at a fluorescence emission wavelength. The fluorometer 514, in some examples, may also include other optical components used to facilitate the emission and detection of specific wavelengths of light to cause and detect fluorescence, including one or more lenses, one or more filters, a beamsplitter, and the like. For instance, the fluorometer 514 may use a beamsplitter and filters to receive and transmit (and, block) respective wavelengths of light in a common light pathway, which in turn is communicated via the light pipe 530.

The following example explains use of the light pipe 530 in connection with insertion of a portion of the light pipe 530 into an interior chamber of the endoscope 200. In other examples, not illustrated, the inspection device 500 may expose a component of the fluorometer 514 or a channel connected to the fluorometer 514 to allow dispersing and detection of light directly from the inspection device 500 without an attached light pipe. Thus, it will be understood that the inspection device 500 may comprise a variety of handheld or standalone device form factors, including into wand, gun, or probe shapes, which enable one-handed use to perform the rapid examination of multiple surfaces and/or spaces of an endoscope, and identify fluorescence emitted from biological contamination.

The use illustration of FIG. 5 specifically depicts an insertion of a portion of light pipe 530 into an interior chamber of the endoscope 200, and specifically, an insertion of a distal end of the light pipe 530 into a biopsy channel portion of the suction channel 230 that extends along a length of the insertion tube 204. The light pipe 530 includes a tip 536 at a distal end, which permits light to be emitted to and received from the channel 230. The light pipe 530 also includes the connector 532 at a proximate end, which also permits this light to be emitted to and received from the inspection device 500. The light pipe 530 extends along a length of an optical fiber 534 (shortened for illustration purposes) which defines a light pathway that allows the light to pass in both directions. The optical fiber 534 and the light pipe 530 may be flexible and shaped and sized to permit passage in the lumen of the endoscope.

As shown, the insertion of the light pipe 530 into the biopsy valve 214 and the channel 230 may allow the identification of fluorescence from biological material such as proteins (illustrated by material deposit 240) remaining after manual cleaning and flushing. For instance, light emitted at a fluorescence excitation wavelength via the light pipe 530 may be used to cause fluorescence of biological material exposed to a fluorescing agent. In turn, light sensed at a fluorescence emission wavelength may be observed from the fluorescence of the biological material in the presence of the fluorescing agent. In a specific example, the fluorescence excitation wavelength comprises a wavelength (in air) of 300-390 nm (or more specifically, 330-390 mm), and the fluorescence emission wavelength comprises a wavelength (in air) of 400-475 nm (or more specifically, 436-475 nm).

The fluorescing agent may comprise any number of chemical agents or compositions, including those designed for other uses in the cleaning workflow. In an example, the fluorescing agent is an additive to rinse water, or applied as a bedside cleaning solution, so that contamination or ingrained biofilm will become stained by the time the endoscope begins manual reprocessing. In a further example, the fluorescing agent may be a component of a decontamination foam. In a further example, the fluorescing agent is a composition comprising ortho-phthalaldehyde (OPA), a high level disinfectant ingredient. In a more specific example, the fluorescing agent is a cleaning composition comprising an alkaline detergent combined with a high-level disinfectant comprising ortho-phthalaldehyde.

The fluorescence inspection device 500, though operation of the fluorometer 514, may indicate the presence of detectable light at the fluorescence emission wavelength (e.g., the detection of the light at the wavelength above a certain detectable threshold); in other examples, the fluorometer 514 may measure and indicate a level of the light sensed at the fluorescence emission wavelength (e.g., the amount or intensity of the light at the fluorescence emission wavelength). The detection or measurement may be correlated to produce an indicator of a detection of the fluorescence emission wavelength (or, a detection of a lack of the fluorescence emission wavelength) from the inspection device 500. For instance, this may be provided as an output on the indication device 502, a storage of a data value within the inspection device 500, or communication to an external system (not shown).

Also in further examples, the use of the fluorescence device may be integrated with a borescope and borescope inspection system. For instance, a borescope which is designed for visual inspection of an endoscope lumen may also include components that enable inspection for fluorescence. Such fluorescence may be observed as a result of the reaction of residual biological material which has reacted to fluorescing agents, such as after a disinfectant flush and/or rinse. Such fluorescence may also be observed in a lumen which includes a first functional layer and a second fluorescing layer which indicates that the first functional layer has been damaged, scratched, broken, etc. if fluorescing is visible. The integration of the fluorescence device with a borescope may be provided through the use of a light pipe or optical fiber extending along the length of the borescope; or specialized light emitting and/or light sensing components which accompany the optical camera of the borescope. Thus, any number of optical and fluorescing inspection devices and probes may be adapted for use of the present techniques.

Also in further examples, the use of the fluorescence device or the light pipe may be integrated with features of an optical magnifier or like visual inspection tool, to indicate fluorescence in relation to a visually inspected area. In particular, a fluorescence detection capability may be integrated into a handheld magnifier that provides a multi-purpose tool for the inspection of the external surfaces of the endoscope.

Also in further examples, the use of the fluorescence device may be integrated with features of an automated cleaning machine or machine-based rinsing, cleaning, or disinfection system. The fluorescence device may perform analysis of a fluid from a machine to detect and indicate the amount of residual contamination from proteins or other biological materials remaining in fluid after use of a cleaning or rinsing solution. Moreover, for instance, such analysis may first occur from fluid exiting the endoscope during a manual cleaning stage with the flushing aid, or, may also occur with monitoring during the cleaning stage of an AER cycle. Such monitoring may include real time monitoring after the self-disinfection cycle of an AER, to confirm the efficacy of the cleaning process.

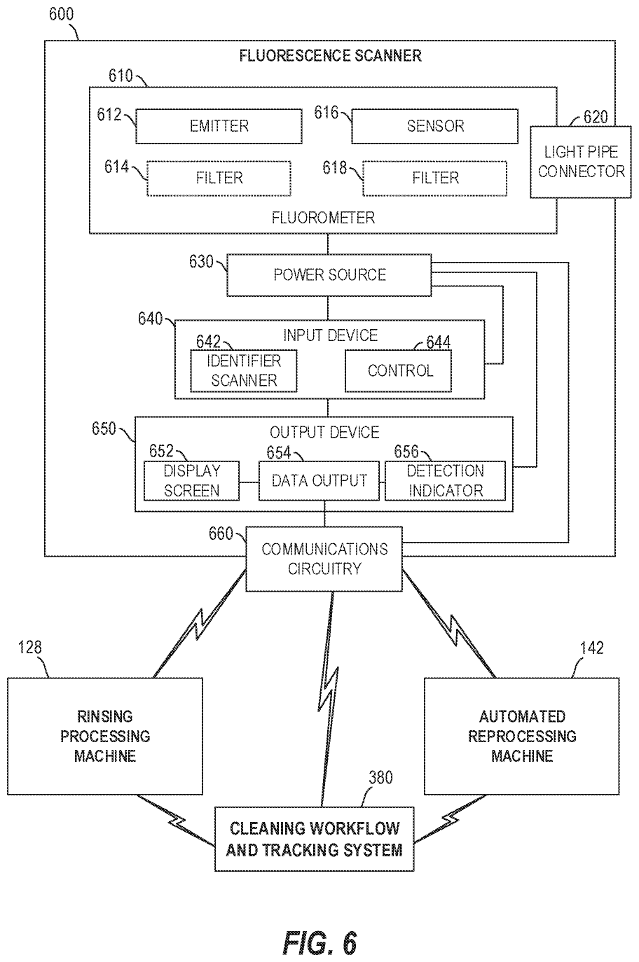

FIG. 6 is a block diagram that illustrates further components and integration of an example fluorescence inspection system 600. This system may be embodied by the configuration and use cases of fluorescence inspection devices 126, 500 discussed above; the system alternatively may be embodied by other form factors and use cases involving other types of inspection scenarios, circuitry, or components.

The fluorescence inspection system 600 is depicted as including a fluorometer 610, a light pipe connector 620, a power source 630, an input device 640, an output device 650, and communications circuitry 660. The indication of a contamination detection obtained via fluorescence may be communicated from the fluorescence inspection system 600 to other devices and systems used in connection with cleaning, such as a rinsing processing machine 128, an automated reprocessing machine 142, and/or a cleaning workflow and tracking system 380. For instance, a fluorescence scanner may be adapted to be operable by a human technician during manual cleaning operations, while automatically communicating a status of any detected fluorescence (detected contamination from biological materials) to a cleaning workflow and tracking system. The fluorescence inspection system 600 may also be adapted for analysis of fluorescence levels in fluids, such as disinfectant or rinsing fluids, as the levels are automatically communicated to a rinsing processing machine 128. The fluorescence inspection system 600 may also be adapted for analysis of fluorescence levels or presence in surfaces or areas of an endoscope, as the levels or presence of fluorescence and derived contamination is communicated to an automated reprocessing machine 142. Further integration of data from the fluorescence inspection system 600 as part of tracking and cleaning operations may also follow the examples discussed above.

The fluorometer 610 is depicted as including an emitter 612, used to emit light at a fluorescence excitation wavelength from the fluorometer 610, and a sensor 616, used to sense light at a fluorescence emission wavelength at the fluorometer. In an example, the fluorometer 610 is a filter fluorometer that includes the emitter 612 and the sensor 616, with use of a primary filter 614 coupled to the emitter 612 to allow passage of the fluorescence excitation wavelength or wavelengths (e.g., block other wavelengths), and a secondary filter 618 coupled to the sensor 616 to allow passage of the fluorescence emission wavelength (e.g., block other wavelengths, including blocking the fluorescence excitation wavelength or wavelengths). In other examples, the emitter 612 and sensor 616 include specific properties to be tuned to the specific fluorescence wavelengths. The fluorometer 610 may integrate with a light pipe connector 620 to allow connection to a light pipe such as a flexible optical fiber. The fluorometer 610 may receive power from a power source 630, and be integrated with other control or communication components (not depicted).

The power source 630 may comprise a removable or fixed battery or power line, including in connection to an external power source. The power source 630 may provide power to electronic processing or input/output components such as the input device 640, the output device 650, and/or the communications circuitry 660.

The input device 640 is depicted as including an identifier scanner 642 and a control component 644. In an example, the identifier scanner 642 is adapted to receive an identification of an endoscope, such as with use of an RFID interrogator to obtain the identifier from an RFID tag of the endoscope, or a barcode reader to obtain the identifier from a barcode of the endoscope. The identifier may represent a make, model, serial number, or other relevant details of the endoscope. The data produced by the fluorometer 610 may be associated, combined, or otherwise tracked based on the identifier or other determined identification of the endoscope. The control component 644 may also allow operative settings of the fluorescence inspection system 600 to be established or modified by a user, such as settings involving a threshold detection level, types or forms of indications, and the like.

The output device 650 is depicted as including a display screen 652, data output component 654, and detection indicator 656. One or more of these output components may be presented based on the form factor, type, and use cases for the fluorescence inspection system 600. For instance, in one example, the display screen 652 may provide a graphical output; in other examples, the display screen 652 may be replaced with one or more LEDs which output the indication of whether contamination is detected or not detected (or, has been detected within a period of time or state). The data output component 654 may comprise a data output port (e.g., USB port, serial port, etc.) used to export data values. The detection indicator 656 may comprise LEDs with basic status of detection in simplest use cases, or graphical and text outputs provided for output via the display screen 652 in more complex use cases. The output device 650 may also operate using other forms of circuitry and logic.

Finally, the fluorescence inspection system 600 is depicted as including the communications circuitry 660, which may be used to communicate the indication of fluorescence (contamination) and an identifier of the endoscope to an external system, such as the rinsing processing machine 128, the cleaning workflow and tracking system 380, and/or the automated reprocessing machine 142.

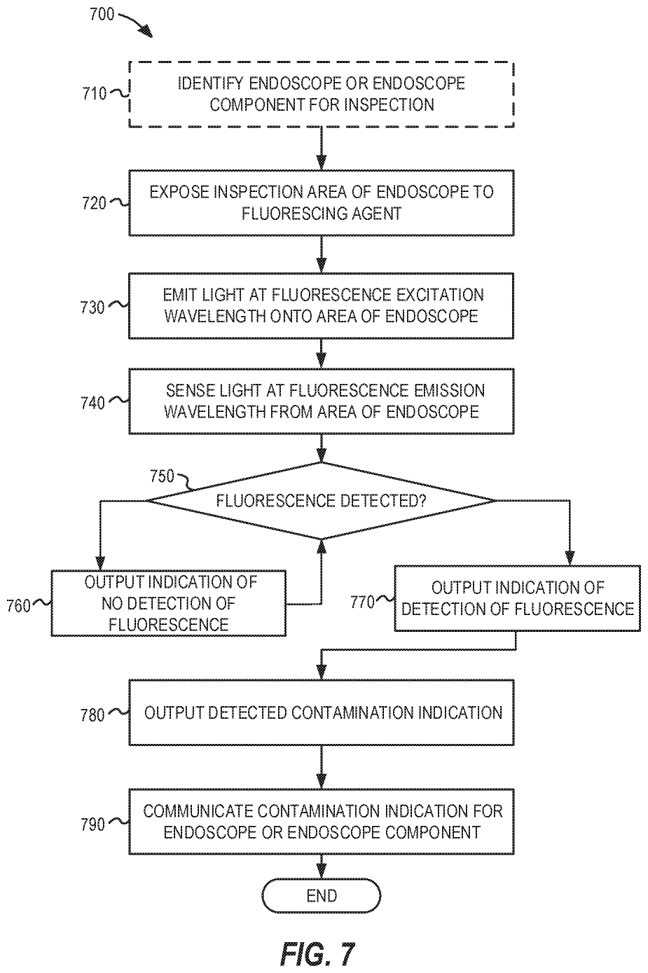

FIG. 7 illustrates a flowchart 700 for a method of endoscope inspection, according to various examples discussed herein. The flowchart 700 is depicted in relation to a sequence which may be performed during an inspection process in a manual cleaning workflow. It will be understood, however, that the depicted operations may occur in other workflows and in other orders.

The flowchart 700 commences at 710 with an optional identification of the endoscope or endoscope component for inspection. This identification information may be used to track the cleaning or contamination status of the endoscope or endoscope component, such as with a cleaning and tracking workflow.

The flowchart 700 continues at 720 by exposing an area of the endoscope to be inspected to a fluorescing agent (e.g., a disinfecting or cleaning agent). This is followed at 730 by the emission of light at a fluorescence excitation wavelength onto the area of the endoscope, and at 740 with the sensing of light at a different fluorescence emission wavelength from the area of the endoscope. The emission and sensing may be performed with the use of the fluorescence inspection system 600 discussed above, the other device embodiments 126, 500, or like systems discussed herein that involve fluorescence inspection.

Based on the evaluation 750 of whether fluorescence is detected, an indication is output at 760, if no fluorescence is detected, to identify no detection (a lack) of fluorescence. If fluorescence is detected, an indication is output at 770 to identify the detection of fluorescence. This indication may be combined or followed at 780 with an output of an indication of detected contamination for the area of the endoscope. For instance, this contamination indication may occur in real-time on an inspection device, to provide instant feedback that the area being inspected has fluorescence detected from residual biological materials.

Finally, the flowchart 700 concludes at 790 with the communication of the contamination indication for the area of the endoscope being inspected. This contamination indication may be communicated via any number of electronic mechanisms to output devices, communication systems, databases, or external systems, as discussed in the examples above.

Although many of the preceding examples were provided with reference to endoscope processing and similar medical device cleaning settings, it will be understood that a variety of other uses may be applied in both medical and non-medical settings to identify, prevent, or reduce the potential of contamination. These settings may include the handling of hazardous materials in a various of scientific and industrial settings, such as the handling of objects contaminated with biological or radioactive agents; the human control of systems and devices configured to process and clean potentially contaminated objects; and other settings involving a contaminated object or human. Likewise, the preceding examples may also be applicable in clean room settings where the environment or particular objects are intended to remain in a clean state, and where human contact with substances or objects may cause contamination that is tracked and remediated.

As an additional example, computing embodiments described herein may be implemented in one or a combination of hardware, firmware, and software. Embodiments may also be implemented as instructions or executable stored on a computer-readable storage device, which may be read and executed by at least one processor to perform the operations described herein. A computer-readable storage device may include any non-transitory mechanism (e.g., a non-transitory machine-readable storage medium) for storing information in a form readable by a machine (e.g., a computer). For example, a computer-readable storage device may include read-only memory (ROM), random-access memory (RAM), magnetic disk storage media, optical storage media, flash-memory devices, and other storage devices and media. Other electronic device components including embedded logic circuity, system-on-chip (SoC) circuitry, and other forms of processing circuitry may also embody or implement such instructions or executable logic.

It should be understood that the functional units or capabilities described in this specification may have been referred to or labeled as components or modules, in order to more particularly emphasize their implementation independence. Component or modules may be implemented in any combination of hardware circuits, programmable hardware devices, other discrete components. Components or modules may also be implemented in software for execution by various types of processors. An identified component or module of executable code may, for instance, comprise one or more physical or logical blocks of computer instructions, which may, for instance, be organized as an object, procedure, or function. Nevertheless, the executables of an identified component or module need not be physically located together, but may comprise disparate instructions stored in different locations which, when joined logically together, comprise the component or module and achieve the stated purpose for the component or module. Indeed, a component or module of executable code may be a single instruction, or many instructions, and may even be distributed over several different code segments, among different programs, and across several memory devices.

Similarly, operational data may be identified and illustrated herein within components or modules, and may be embodied in any suitable form and organized within any suitable type of data structure. The operational data may be collected as a single data set, or may be distributed over different locations including over different storage devices, and may exist, at least partially, merely as electronic signals on a system or network. The components or modules may be passive or active, including agents operable to perform desired functions.

Additional examples of the presently described method, system, and device embodiments include the following, non-limiting configurations. Each of the following non-limiting examples may stand on its own, or may be combined in any permutation or combination with any one or more of the other examples provided below or throughout the present disclosure.

Example 1 is an inspection device, comprising: a housing; an optical fiber external to the housing, the optical fiber shaped and sized to permit passage in a lumen of an endoscope; a fluorescence device internal to the housing, the fluorescence device operably coupled to the optical fiber, and the fluorescence device comprising: an emitter to emit excitation light that includes, a fluorescence excitation wavelength into the optical fiber; and a sensor to sense fluorescent light that includes a fluorescence emission wavelength from the optical fiber; and an output device exposed from the housing, the output device configured to output an indication in response to a detection of the fluorescent light with the fluorescence device.

In Example 2, the subject matter of Example 1 includes, subject matter where the output device is further configured to output a second indication in response to a lack of detection of the fluorescent light.

In Example 3, the subject matter of Examples 1-2 includes, subject matter where the fluorescence device is further configured to detect the presence of the fluorescent light.

In Example 4, the subject matter of Examples 1-3 includes, subject matter where the fluorescence device is further configured to measure a level of the fluorescent light, and wherein the indication represents the level of the fluorescent light.

In Example 5, the subject matter of Examples 1-4 includes, subject matter where the optical fiber is coupled to the fluorescence device via an attachment coupler, to allow removable detachment of the optical fiber from the fluorescence device.

In Example 6, the subject matter of Examples 1-5 includes, subject matter where the lumen of the endoscope is hosted in an internal channel that extends from an opening at a distal end of an insertion tube of the endoscope along at least a portion of the length of the insertion tube.

In Example 7, the subject matter of Examples 1-6 includes, subject matter where the emitter comprises a light emitting diode adapted to emit the excitation light.

In Example 8, the subject matter of Examples 1-7 includes, subject matter where the fluorescence device is a filter fluorometer that includes the emitter and the sensor, the filter fluorometer further including a primary filter coupled to the emitter to allow passage of the fluorescence excitation wavelength, and a secondary filter coupled to the sensor to block passage of the fluorescence excitation wavelength and allow passage of the fluorescence emission wavelength.

In Example 9, the subject matter of Examples 1-8 includes, subject matter where the output device comprises: a display screen, a light emitting diode, or a measurement indicator device.

In Example 10, the subject matter of Examples 1-9 includes, subject matter where the excitation light causes fluorescence of a biological material exposed to a fluorescing agent, and wherein the fluorescent light is produced by the fluorescence of the biological material in the presence of the fluorescing agent.

In Example 11, the subject matter of Example 10 includes, subject matter where the fluorescing agent is a cleaning composition comprising an alkaline detergent combined with a high-level disinfectant comprising ortho-phthalaldehyde.

In Example 12, the subject matter of Examples 10-11 includes, subject matter where the fluorescing agent is a composition comprising ortho-phthalaldehyde.

In Example 13, the subject matter of Examples 1-12 includes, subject matter where the fluorescence excitation wavelength is between 330 nm and 390 nm, inclusive, and wherein the fluorescence emission wavelength is between 436 nm and 475 nm, inclusive.

In Example 14, the subject matter of Examples 1-13 includes, communication circuitry to communicate an indication of the detection of the fluorescence emission wavelength to a computing device.

In Example 15, the subject matter of Examples 1-14 includes, an input device, the input device configured to receive input that identifies the endoscope.

In Example 16, the subject matter of Examples 1-15 includes, an identifier scanner, to obtain an identifier of the endoscope.

In Example 17, the subject matter of Example 16 includes, subject matter where the identifier scanner is an RFID interrogator usable to obtain the identifier from an RFID tag of the endoscope.

In Example 18, the subject matter of Examples 16-17 includes, subject matter where the identifier scanner is a barcode reader usable to obtain the identifier from a barcode of the endoscope.

In Example 19, the subject matter of Examples 16-18 includes, a data output device, the data output device to provide an output of the indication of the detection of the fluorescence emission wavelength as associated with the identifier of the endoscope.

In Example 20, the subject matter of Examples 16-19 includes, communication circuitry to communicate the indication of the detection of the fluorescence emission wavelength and the identifier of the endoscope to a tracking system.

Example 21 is a method of endoscope inspection, comprising: emitting excitation light at a fluorescence excitation wavelength, using a fluorometer, onto an area of an endoscope; sensing fluorescent light at a fluorescence emission wavelength, using the fluorometer, from the area of the endoscope; and outputting an indication, with an output device, in response to sensing the fluorescent light.

In Example 22, the subject matter of Example 21 includes, subject matter where the area of the endoscope comprises a lumen of a channel of the endoscope.

In Example 23, the subject matter of Example 22 includes, subject matter where the excitation light is emitted to the lumen of the channel of the endoscope from the fluorometer via an optical fiber, the optical fiber being shaped and sized to permit passage in the channel of the endoscope.

In Example 24, the subject matter of Example 23 includes, subject matter where the optical fiber is coupled to the fluorometer via an attachment coupler, to allow removable detachment of the optical fiber.

In Example 25, the subject matter of Examples 21-24 includes, subject matter where the area of the endoscope comprises an exterior surface of the endoscope.

In Example 26, the subject matter of Examples 21-25 includes, subject matter where the fluorometer and the output device are integrated into a device form factor operable for handheld use by a human user.

In Example 27, the subject matter of Examples 21-26 includes, outputting a second indication in response to a lack of detection of the fluorescent light.

In Example 28, the subject matter of Examples 21-27 includes, measuring an amount of the sensed fluorescent light.

In Example 29, the subject matter of Examples 21-28 includes, outputting a second indication in response to a lack of detection of the fluorescent light.

In Example 30, the subject matter of Examples 21-29 includes, subject matter where sensing the fluorescent light comprises detecting the presence of the fluorescent light, or measure a level of the fluorescent light, wherein the indication represents the presence or the level of the fluorescent light.

In Example 31, the subject matter of Examples 21-30 includes, subject matter where the fluorometer is a filter fluorometer that includes an emitter and a sensor, the filter fluorometer further including a primary filter coupled to the emitter to allow passage of the fluorescence excitation wavelength, and a secondary filter coupled to the sensor to block passage of the fluorescence excitation wavelength and allow passage of the fluorescence emission wavelength.

In Example 32, the subject matter of Examples 21-31 includes, subject matter where the output device comprises: a display screen, a light emitting diode, or a measurement indicator device.

In Example 33, the subject matter of Examples 21-32 includes, subject matter where the excitation light causes fluorescence of a biological material exposed to a fluorescing agent, and wherein the fluorescent light is produced by the fluorescence of the biological material in the presence of the fluorescing agent.

In Example 34, the subject matter of Example 33 includes, subject matter where the fluorescing agent is a cleaning composition comprising an alkaline detergent combined with a high-level disinfectant comprising ortho-phthalaldehyde.

In Example 35, the subject matter of Examples 33-34 includes, subject matter where the fluorescing agent is a composition comprising ortho-phthalaldehyde.

In Example 36, the subject matter of Examples 21-35 includes, subject matter where the fluorescence excitation wavelength is between 330 nm and 390 nm, inclusive, and wherein the fluorescence emission wavelength is between 436 nm and 475 nm, inclusive.

In Example 37, the subject matter of Examples 21-36 includes, communicating the indication of sensing the fluorescent light to a computing device.

In Example 38, the subject matter of Examples 21-37 includes, obtaining input that identifies the endoscope using an input device.

In Example 39, the subject matter of Examples 21-38 includes, obtaining an identifier of the endoscope using an identifier scanner.

In Example 40, the subject matter of Example 39 includes, subject matter where the identifier scanner is an RFID interrogator adapted to obtain the identifier from an RFID tag of the endoscope, or a barcode reader adapted to obtain the identifier from a barcode of the endoscope.

In Example 41, the subject matter of Examples 39-40 includes, communicating the indication and the identifier of the endoscope to a tracking system.

Example 42 is an inspection device, comprising: a housing; a power source integrated within the housing; a fluorometer integrated within the housing and operably coupled to the power source, the fluorometer comprising: an emitter exposed from the housing to emit excitation light that includes, a fluorescence excitation wavelength to a surface of a reusable medical instrument; and a sensor exposed from the housing to sense fluorescent light that includes a fluorescence emission wavelength from the surface of the reusable medical instrument; and an output device exposed from the housing and operably coupled to the power source, the output device configured to output an indication in response to a detection of the fluorescent light with the fluorometer.

In Example 43, the subject matter of Example 42 includes, a connector to removably couple to a proximate end of an optical fiber, wherein the emitter and sensor emit and sense light via the connector and the optical fiber, to allow emission of the excitation light and the sensing of the fluorescent light via a distal end of the optical fiber.

The following claims are hereby incorporated into the detailed description, with each claim standing on its own as a separate embodiment.

What is claimed is:

1. An inspection device, comprising:
a housing;
an optical fiber external to the housing, the optical fiber shaped and sized to permit passage in a lumen of an endoscope;
a fluorescence device internal to the housing, the fluorescence device operably coupled to the optical fiber, and the fluorescence device comprising:
an emitter to emit excitation light that includes a fluorescence excitation wavelength into the optical fiber; and
a sensor to sense fluorescent light that includes a fluorescence emission wavelength from the optical fiber; and
an output device exposed from the housing, the output device configured to output an indication in response to a detection of the fluorescent light with the fluorescence device.

2-6. (canceled)

7. The inspection device of claim 1, wherein the emitter comprises a light emitting diode adapted to emit the excitation light.

8. The inspection device of claim 1, wherein the fluorescence device is a filter fluorometer that includes the emitter and the sensor, the filter fluorometer further including a primary filter coupled to the emitter to allow passage of the fluorescence excitation wavelength, and a secondary filter coupled to the sensor to block passage of the fluorescence excitation wavelength and allow passage of the fluorescence emission wavelength.

9. The inspection device of claim 1, wherein the output device comprises: a display screen, a light emitting diode, or a measurement indicator device.

10. The inspection device of claim 1, wherein the excitation light causes fluorescence of a biological material exposed to a fluorescing agent, and wherein the fluorescent light is produced by the fluorescence of the biological material in the presence of the fluorescing agent.

11. The inspection device of claim 10, wherein the fluorescing agent is a cleaning composition comprising an alkaline detergent combined with a high-level disinfectant comprising ortho-phthalaldehyde.

12-15. (canceled)

16. The inspection device of claim 1, further comprising an identifier scanner, to obtain an identifier of the endoscope.

17-19. (canceled)

20. The inspection device of claim 16, further comprising communication circuitry to communicate the indication of the detection of the fluorescence emission wavelength and the identifier of the endoscope to a tracking system.

21. A method of endoscope inspection, comprising:
emitting excitation light at a fluorescence excitation wavelength, using a fluorometer, onto an area of an endoscope, wherein the fluorometer is integrated into an inspection device having a form factor operable for handheld use by a human user;
sensing fluorescent light at a fluorescence emission wavelength, using the fluorometer, from the area of the endo scope; and
outputting an indication, with an output device, in response to sensing the fluorescent light.

22. The method of claim 21, wherein the area of the endoscope comprises a lumen of a channel of the endoscope.

23. The method of claim 22, wherein the excitation light is emitted to the lumen of the channel of the endoscope from the fluorometer via an optical fiber coupled to the inspection device, the optical fiber being shaped and sized to permit passage in the channel of the endoscope.

24. The method of claim 23, wherein the optical fiber is coupled to the inspection device via an attachment coupler, to allow removable detachment of the optical fiber.

25-33. (canceled)

34. The method of claim 21, wherein the fluorescing agent is a cleaning composition comprising an alkaline detergent combined with a high-level disinfectant comprising ortho-phthalaldehyde.

35. The method of claim 21, wherein the fluorescing agent is a composition comprising ortho-phthalaldehyde.

36. (canceled)

37. (canceled)

38. The method of claim 21, further comprising, obtaining input that identifies the endoscope using an input device.

39. The method of claim 21, further comprising, obtaining an identifier of the endoscope using an identifier scanner.

40. The method of claim 39, wherein the identifier scanner is an RFID interrogator adapted to obtain the identifier from an RFID tag of the endoscope, or a barcode reader adapted to obtain the identifier from a barcode of the endoscope.

41. The method of claim 39, further comprising, communicating the indication and the identifier of the endoscope to a tracking system.

42. An inspection device, comprising:
a housing;
a power source integrated within the housing;
a fluorometer integrated within the housing and operably coupled to the power source, the fluorometer comprising:
an emitter exposed from the housing to emit excitation light that includes a fluorescence excitation wavelength to a surface of a reusable medical instrument; and
a sensor exposed from the housing to sense fluorescent light that includes a fluorescence emission wavelength from the surface of the reusable medical instrument; and
an output device exposed from the housing and operably coupled to the power source, the output device configured to output an indication in response to a detection of the fluorescent light with the fluorometer.

43. The inspection device of claim 42, further comprising:
a connector to removably couple to a proximate end of an optical fiber, wherein the emitter and sensor emit and sense light via the connector and the optical fiber, to allow emission of the excitation light and the sensing of the fluorescent light via a distal end of the optical fiber.
